(19) United States
(12) Patent Application Publication
(10) Pub. No.: US 2022/0176285 A1
(43) Pub. Date: Jun. 9, 2022

(54) FILTRATION FILTER

(71) Applicant: Murata Manufacturing Co., Ltd., Nagaokakyo-shi (JP)

(72) Inventors: Shusuke YOKOTA, Nagaokakyo-shi (JP); Takashi KONDO, Nagaokakyo-shi (JP)

(21) Appl. No.: 17/652,617

(22) Filed: Feb. 25, 2022

Related U.S. Application Data
(63) Continuation of application No. PCT/JP2020/021785, filed on Jun. 2, 2020.

(30) Foreign Application Priority Data
Aug. 30, 2019 (JP) 2019-158685

(51) Int. Cl. B01D 39/20

(52) U.S. Cl. CPC B01D 39/20; G01N 1/04

(57) ABSTRACT

A filtration filter that filters a liquid containing a filtration target, the filtration filter including a filter base portion defining a plurality of through-holes. The filter base portion includes a plurality of first base portions and a plurality of second base portions that are thinner than the plurality of first base portions. Each of the plurality of second base portions are between the plurality of first base portions.

### CROSS REFERENCE TO RELATED APPLICATIONS

The present application is a continuation of International application No. PCT/JP2020/021785, filed Jun. 2, 2020, which claims priority to Japanese Patent Application No. 2019-158685, filed Aug. 30, 2019, the entire contents of each of which are incorporated herein by reference.

### FIELD OF THE INVENTION

The present invention relates to a filtration filter.

### BACKGROUND OF THE INVENTION

Patent Document 1 discloses a cell capturing metal filter as a filter for capturing cells, for example.

Patent Document 1: Japanese Unexamined Patent Application Publication No. 2015-188323

### SUMMARY OF INVENTION

However, the filter described in Patent Document 1 still has room for improvement in terms of increasing the filtration efficiency.

It is an object of the present invention to provide a filtration filter capable of increasing filtration efficiency.

A filtration filter according to one aspect of the present invention is a filtration filter for filtering a liquid containing a filtration target, the filtration filter including a filter base portion defining a plurality of through-holes, wherein the filter base portion includes a plurality of first base portions and a plurality of second base portions that are thinner than the plurality of first base portions, and each of the plurality of second base portions are between the plurality of first base portions.

According to the present invention, it is possible to provide a filtration filter capable of increasing filtration efficiency.

### BRIEF DESCRIPTION OF THE DRAWINGS

FIG. 1 is a schematic view of an example of a filtration filter of Embodiment 1 according to the present invention.

FIG. 2 is an enlarged schematic view of a portion of a filter portion in FIG. 1.

FIG. 3 is a schematic sectional view of the filter portion in FIG. 2 taken along a line A-A.

FIG. 4A is a schematic diagram of an example of a process in a manufacturing method of the filtration filter of Embodiment 1 according to the present invention.

FIG. 4B is a schematic diagram of an example of a process in the manufacturing method of the filtration filter of Embodiment 1 according to the present invention.

FIG. 4C is a schematic diagram of an example of a process in the manufacturing method of the filtration filter of Embodiment 1 according to the present invention.

FIG. 4D is a schematic diagram of an example of a process in the manufacturing method of the filtration filter of Embodiment 1 according to the present invention.

FIG. 4E is a schematic diagram of an example of a process in the manufacturing method of the filtration filter of Embodiment 1 according to the present invention.

FIG. 4F is a schematic diagram of an example of a process in the manufacturing method of the filtration filter of Embodiment 1 according to the present invention.

### DETAILED DESCRIPTION OF THE PREFERRED EMBODIMENTS

### Background of the Present Invention

In the filter described in Patent Document 1, the main surface of the filter is formed in a flat shape. With this, the liquid containing the filtration target dropped on the main surface of the filter hardly spreads on the main surface of the filter, and the filtration area actually used for filtration in the area of the entire filter is small.

The inventors of the present invention examined to increase filtration efficiency by increasing a filtration area of a filter during filtration. As a result, the inventors of the present invention have found to partially provide a portion having a small thickness in a filter increases filtration efficiency. Thus, the present invention has been conceived.

A filtration filter according to one aspect of the present invention is a filtration filter for filtering a liquid containing a filtration target, the filtration filter including a filter base portion defining a plurality of through-holes, wherein the filter base portion includes a plurality of first base portions and a plurality of second base portions that are thinner than the plurality of first base portions, and each of the plurality of second base portions are between the plurality of first base portions.

With the configuration above, the filtration efficiency may be increased.

The width of the plurality of second base portions may be larger than the width of the plurality of first base portions.

With the configuration above, the filtration efficiency may be further increased.

The plurality of first base portions and the plurality of second base portions may be in a grid pattern.

With the configuration above, the filtration efficiency may be further increased.

The plurality of second base portions may be periodically arranged.

With the configuration above, the filtration efficiency may be further increased.

The filtration filter may include a reinforcement layer on the filter base portion.

With the configuration above, the strength of the filtration filter may be increased.

The filter base portion may comprise at least one of a metal and a metal oxide as a main component.

With the configuration above, the filtration efficiency may be further increased.

Hereinafter, Embodiment 1 according to the present invention will be described with reference to the accompanying drawings. In the drawings, each element is appropriately emphasized for facilitating the description.

### Embodiment 1

A filtration filter of Embodiment 1 according to the present invention is a filter for filtering a liquid containing a filtration target.

In this description, the “filtration target” means an object to be filtered among objects contained in a liquid. The filtration target may be a biological substance contained in a liquid, for example. “Biological substance” means a substance originated in an organism such as a cell (eukaryote), a bacterium (eubacterium), or a virus. Examples of cells (eukaryotes) include artificial pluripotent stem cells (iPS cells), ES cells, stem cells, mesenchymal stem cells, mononuclear cells, single cells, cell masses, planktonic cells, adherent cells, nerve cells, leukocytes, regenerative medical cells, autologous cells, cancer cells, circulating cancer cells (CTCs), HL-60, HELA, and fungi. Examples of the bacteria (eubacteria) include Escherichia coli and Mycobacterium tuberculosis.

In Embodiment 1, there will be described an example in which the liquid is a cell suspension and the filtration target is a cell.

[Overall Configuration]

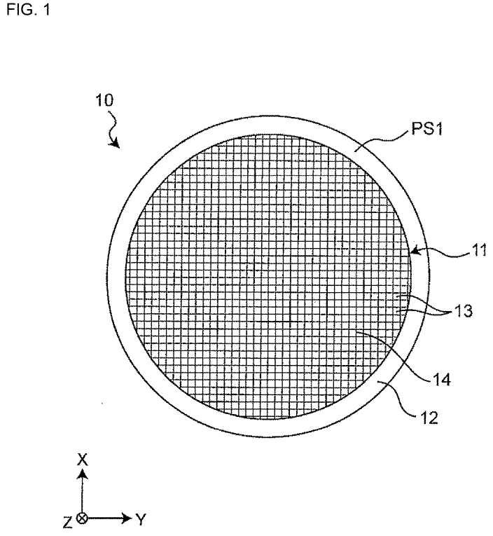

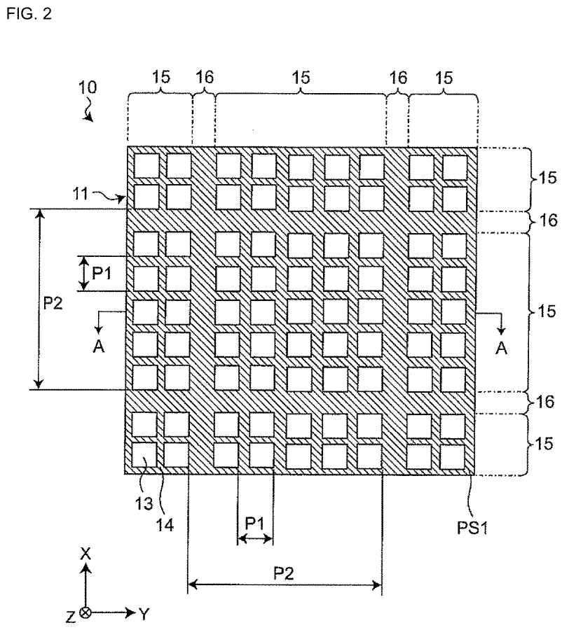

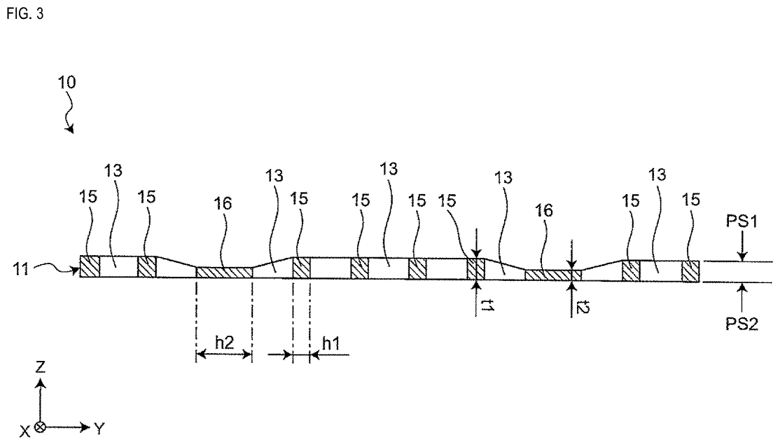

FIG. 1 is a schematic view of an example of a filtration filter 10 of Embodiment 1 according to the present invention. FIG. 2 is an enlarged schematic view of a portion of a filter portion 11 in FIG. 1. FIG. 3 is a schematic sectional view of the filter portion 11 in FIG. 2 taken along a line A-A. The X, Y, and Z directions in the drawings respectively indicate the longitudinal direction, the lateral direction, and the thickness direction of the filtration filter 10.

As illustrated in FIG. 1, the filtration filter 10 includes the filter portion 11 having a plurality of through-holes and a frame portion 12 disposed so as to surround the outer periphery of the filter portion 11. In Embodiment 1, the filter portion 11 and the frame portion 12 are integrally formed.

The filtration filter 10 is a metal filter. A material constituting the filtration filter 10 contains at least one of metal and metal oxide as a main component. The material constituting the filtration filter 10 may be gold, silver, copper, platinum, nickel, palladium, titanium, alloys thereof, or oxides thereof, for example. In particular, using titanium or a nickel-palladium alloy makes the elution of metal small, and influence on the filtration target may be reduced.

The filtration filter 10 is a plate-shaped structure having a first main surface PS1 and a second main surface PS2 facing the first main surface PS1. A filtration target contained in a liquid is captured on the first main surface PS1.

<Filter Portion>

As illustrated in FIG. 2 and FIG. 3, the filter portion 11 has a plurality of through-holes 13. Specifically, the plurality of through-holes 13 penetrating through the first main surface PS1 and the second main surface PS2 is formed in the filter portion 11. The filter portion 11 is formed with a filter base portion 14. The filter base portion 14 defines the plurality of through-holes 13.

The shape of the filter portion 11 is circular, rectangular, or elliptical when viewed from the thickness direction (Z direction) of the filtration filter 10, for example. In Embodiment 1, the shape of the filter portion 11 is substantially circular. In the present description, “substantially circular” means that the ratio of the length of the major axis to the length of the minor axis is 1.0 to 1.2.

The plurality of through-holes 13 are periodically arranged on the first main surface PS1 and the second main surface PS2 of the filter portion 11. Specifically, the plurality of through-holes 13 are provided at regular intervals in a matrix shape in the filter portion 11.

The size of the through-hole 13 is appropriately designed in accordance with the type (size, form, property, elasticity) or quantity of the cells that are the filtration target. The aperture ratio of the filter portion 11 is 10% or more, and preferably 25% or more. With the configuration above, it is possible to reduce the passage resistance of the liquid through the filter portion 11. As a result, the processing time may be shortened and the stress on the cells may be reduced. Note that the aperture ratio is calculated as (area occupied by the through-holes 13)/(projected area of the first main surface PS1 on the assumption that the through-holes 13 are not present).

In Embodiment 1, the through-hole 13 has a square shape when viewed from the side of the first main surface PS1 of the filter portion 11, that is, the Z direction. The shape of the through-hole 13 when viewed from the Z direction is not limited to a square, and may be a rectangle, a circle, or an ellipse, for example.

In Embodiment 1, the shape (sectional shape) of the through-hole 13 projected onto a plane perpendicular to the first main surface PS1 of the filter portion 11 is a rectangle. Specifically, the sectional shape of the through-hole 13 is a rectangle in which the length of one side in the radial direction of the filtration filter 10 is longer than the length of one side in the thickness direction of the filtration filter 10. The sectional shape of the through-hole 13 is not limited to a rectangle, and may be a parallelogram or a tapered shape such as a trapezoid, a symmetrical shape, or an asymmetrical shape, for example.

In Embodiment 1, the plurality of through-holes 13 are provided in two arrangement directions parallel to each side of a square when viewed from the side of the first main surface PS1 (Z direction) of the filter portion 11, that is, the X direction and the Y direction in FIG. 2. It is sufficient that the plurality of through-holes 13 are provided in the filter portion 11, and the arrangement direction thereof is not limited.

In the filter portion 11, it is preferable that the first main surface PS1, with which a liquid containing a filtration target comes into contact, have a small surface roughness. Here, the surface roughness means an average value of differences between maximum and minimum values measured at any five points on the first main surface PS1 by a stylus profilometer. In Embodiment 1, the surface roughness is preferably smaller than the size of a filtration target, and more preferably smaller than half the size of a filtration target.

In the filter portion 11, a portion where the through-hole 13 is not formed is formed by the filter base portion 14. As illustrated in FIG. 2 and FIG. 3, the filter base portion 14 includes a plurality of first base portions 15 and a plurality of second base portions 16.

The first base portion 15 is formed of a rectangular bar-shaped member. The plurality of first base portions 15 extends in a plurality of directions and intersects with each other to define the plurality of through-holes 13. In Embodiment 1, the plurality of first base portions 15 are provided in a grid pattern.

Specifically, the plurality of first base portions 15 include a base portion extending in the longitudinal direction (X direction) of the filtration filter 10 and a base portion extending in the lateral direction (Y direction) of the filtration filter 10. The plurality of first base portions 15 are provided at regular intervals P1 in the longitudinal direction (X direction) and the lateral direction (Y direction) of the filtration filter 10 except for a portion connected to the second base portion 16. Further, the plurality of first base portions 15 are periodically arranged over the entire filter portion 11.

The second base portion 16 forms a portion of the filter portion 11 where the thickness of the filtration filter 10 is smaller than that of the first base portion 15. The second base portion 16 is formed of a rectangular bar-shaped member. Each of the plurality of second base portions 16 are provided between the plurality of first base portions 15. The plurality of second base portions 16 extend in a plurality of directions, intersects with each other, and define the plurality of through-holes 13 together with the plurality of first base portions 15. In Embodiment 1, the plurality of second base portions 16 are provided in a grid pattern.

Specifically, the plurality of second base portions 16 include a base portion extending in the longitudinal direction (X direction) of the filtration filter 10 and a base portion extending in the lateral direction (Y direction) of the filtration filter 10. The plurality of second base portions 16 are provided at regular intervals P2 in the longitudinal direction (X direction) and the lateral direction (Y direction) of the filtration filter 10. Further, the plurality of second base portions 16 are periodically arranged over the entire filter portion 11.

In the filtration filter 10, the interval P2 between the plurality of second base portions 16 is larger than the interval P1 between the plurality of first base portions 15. As a result, each of the plurality of second base portions 16 are provided so as to be sandwiched by the plurality of first base portions 15.

In Embodiment 1, in each of the longitudinal direction (X direction) and the lateral direction (Y direction) of the filtration filter 10, four first base portions 15 are provided on one side of one second base portion 16, and four first base portions 15 are provided on the other side of one second base portion 16. That is, in each of the longitudinal direction (X direction) and the lateral direction (Y direction) of the filtration filter 10, one second base portion 16 is sandwiched by four first base portions 15 provided on one side and four first base portions 15 provided on the other side. FIG. 2 only shows a portion of the filtration filter 10 of FIG. 1, and thus only shows three of the four sections mentioned above.

In the filtration filter 10, the portion where the second base portion 16 is sandwiched by the plurality of first base portions 15 is periodically arranged over the entire filter portion 11.

As illustrated in FIG. 3, a thickness t2 of the second base portion 16 is smaller than a thickness t1 of the first base portion 15. In other words, the length of the second base portion 16 in the Z direction is smaller than the length of the first base portion 15 in the Z direction. This achieves the height difference between the first base portion 15 and the second base portion 16 on the side of the first main surface PS1 of the filter portion 11. Note that, the first base portion 15 and the second base portion 16 are formed to be flat on the side of the second main surface PS2 of the filter portion 11. With the configuration above, the liquid dropped on the first main surface PS1 of the filter portion 11 may be made to move to the second base portion 16 positioned lower than the first base portion 15. That is, since the liquid flows from the first base portion 15 toward the second base portion 16, it is possible to increase the filtration area in the filter portion 11 that is actually used for filtration.

For example, the thickness t2 of the second base portion 16 is 0.25 times to 0.95 times the thickness t1 of the first base portion 15. Preferably, the thickness t2 of the second base portion 16 is 0.3 times to 0.7 times the thickness t1 of the first base portion 15. With the configuration above, it is possible to make the liquid dropped on the first main surface PS1 of the filter portion 11 move over a wide range while maintaining the strength of the filtration filter 10.

As illustrated in FIG. 3, a width h2 of the second base portion 16 is larger than a width h1 of the first base portion 15. In other words, the length of the second base portion 16 in the X direction and the Y direction is larger than the length of the first base portion 15 in the X direction and the Y direction. With the configuration above, it is possible to make the liquid dropped on the first main surface PS1 of the filter portion 11 move over a further wide range. Further, since the liquid tends to accumulate on the second base portion 16, it is possible to maintain the activity of the cells captured on the second base portion 16.

For example, the width h2 of the second base portion 16 is 1.1 times to 4 times the width h1 of the first base portion 15. Preferably, the width h2 of the second base portion 16 is 1.5 times 3 times the width h1 of the first base portion 15. With the configuration above, it is possible to make the liquid dropped on the first main surface PS1 of the filter portion 11 move over a wide range.

The plurality of first base portions 15 and the plurality of second base portions 16 are integrally formed.

In the filter portion 11, the ratio of the area occupied by the plurality of second base portions 16 is smaller than the ratio of the area occupied by the plurality of first base portions 15. For example, the area occupied by the plurality of second base portions 16 is 0.001 times to 0.8 times the area occupied by the plurality of first base portions 15 in the filter portion 11, when the filter portion 11 is viewed from the Z direction. Preferably, the area occupied by the plurality of second base portions 16 is 0.01 times to 0.5 times the area occupied by the plurality of first base portions 15.

The frame portion 12 is a member disposed to surround the outer periphery of the filter portion 11. The frame portion 12 is formed in a ring shape when viewed from the side of the first main surface PS1 of the filter portion 11. Further, when the filtration filter 10 is viewed from the side of the first main surface PS1, the center of the frame portion 12 coincides with the center of the filter portion 11. That is, the frame portion 12 is formed concentrically with the filter portion 11.

<Frame Portion>

The frame portion 12 functions as a connection portion to connect the filtration filter 10 to a holder that holds the filtration filter 10.

Further, filter information (such as the dimension of the through-hole 13, for example) may be displayed on the frame portion 12. This makes it easy to grasp the filter hole size without measuring the filter hole size or the like again or to distinguish the front and back sides.

In Embodiment 1, the filtration filter 10 is 33 mm in diameter, and is 20 μm in thickness, for example. The filter portion 11 is 20 mm in diameter, and the frame portion 12 is 2.5 mm in width. The filtration filter 10 is not limited to these dimensions and may be made in other dimensions.

In Embodiment 1, the material configuring the frame portion 12 is the same as the material configuring the filter portion 11 (filter base portion 14). Note that, it is not necessary that the material for the frame portion 12 and the material for the filter portion 11 are the same, and they may be different from each other. Further, it is not necessary that the material for the frame portion 12 and the filter portion 11 are integrally formed, and they may be formed of separate members.

[Manufacturing Method of Filtration Filter]

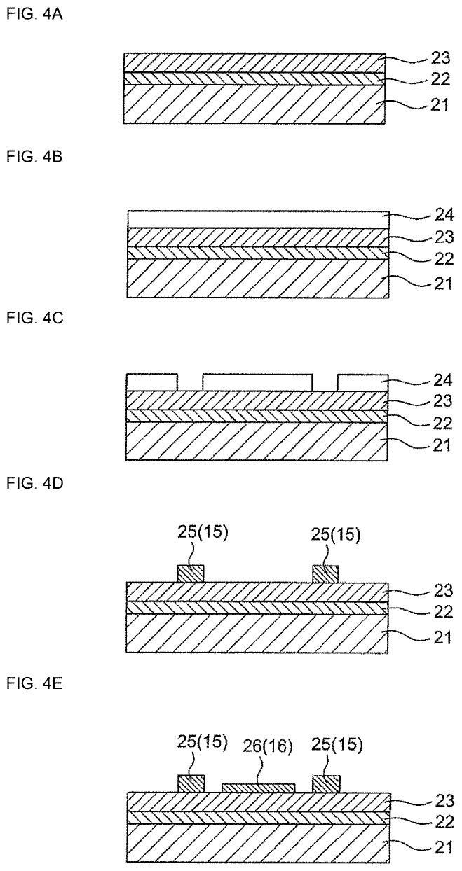

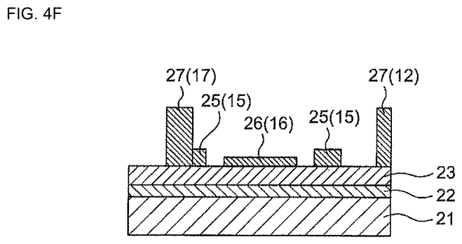

An example of the manufacturing method of the filtration filter 10 will be described with reference to FIG. 4A to FIG. 4F. FIG. 4A to FIG. 4F are schematic diagrams of an example of a process in the manufacturing method of the filtration filter 10 of Embodiment 1 according to the present invention.

As illustrated in FIG. 4A, a copper thin film 22 having the thickness of 500 nm is formed on a substrate 21 made of such as silicon. The copper thin film 22 may be formed with vapor deposition or sputtering. At this time, in order to improve adhesion between the substrate 21 and the copper thin film 22, an intermediate layer 23 of Ti having the thickness of 50 nm is formed.

First, the first base portion 15 is fabricated. As illustrated in FIG. 4B, a resist is applied on the copper thin film 22 with spin coating and dried to form a resist film having the thickness of 2 μm.

As illustrated in FIG. 4C, a resist film 24 is subjected to exposure and development process to remove the resist film 24 at a portion corresponding to the first base portion 15.

PdNi is deposited with electroforming, on the portion from which the resist film 24 is removed. With this, a PdNi plating film is formed on the portion from which the resist film 24 is removed. Subsequently, the resist film 24 is removed using an organic solvent. In this manner, as illustrated in FIG. 4D, the first base portion 15 made of a PdNi plating film 25 is formed.

Next, the second base portion 16 is fabricated by performing the process similar to the process illustrated in FIG. 4A to FIG. 4D. Specifically, a resist film having the thickness of 2 μm is formed on the copper thin film 22 and the intermediate layer 23, on which the first base portion 15 has been fabricated. Next, the resist film is subjected to exposure and development process to remove the resist film at a portion corresponding to the second base portion 16. A PdNi plating film is formed with electroforming, depositing PdNi on the portion from which the resist film is removed. Subsequently, the resist film is removed using an organic solvent. In this manner, as illustrated in FIG. 4E, the second base portion 16 made of a PdNi plating film 26 is formed.

Further, in order to improve the mechanical strength of the filtration filter 10, a reinforcement layer having the grid structure same as that of the filtration filter 10 may be formed. The reinforcement layer may be fabricated by performing a process similar to the process illustrated in FIG. 4A to FIG. 4D.

Specifically, a resist film having the thickness of 30 μm is formed on the copper thin film 22 and the intermediate layer 23, on which the first base portion 15 and the second base portion 16 have been fabricated. Next, the resist film is subjected to exposure and development process to remove the portion of the resist film corresponding to the frame portion 12 and the reinforcement layer. A PdNi plating film is formed with electroforming, depositing PdNi on the portion from which the resist film is removed. Subsequently, the resist film is removed using an organic solvent. With this, as illustrated in FIG. 4F, the frame portion 12 and a reinforcement layer 17 made of a PdNi plating film 27 are formed. Note that, the reinforcement layer 17 was adjusted so as to be disposed on the first base portion 15. The width of the reinforcement layer 17 is 10 μm, and in a case that the width of the first base body portion 15 is smaller than 10 mm, the reinforcement layer 17 is formed so as to straddle the plurality of first base portions 15. Thus, the reinforcement layer 17 is provided on the first base portion 15.

[Effects]

With the use of the filtration filter 10 according to Embodiment 1, the following effects may be achieved.

The filtration filter 10 includes the filter base portion 14 defining the plurality of through-holes 13. The filter base portion 14 includes the plurality of first base portions 15 and the plurality of second base portions 16 thinner than the plurality of first base portions 15. Each of the plurality of second base portions 16 are provided between the plurality of first base portions 15. With the configuration above, the filtration efficiency may be increased.

Further, since each of the plurality of second base portions 16 are provided between the plurality of first base portions 15, the second base portion 16 is sandwiched by at least two first base portions 15. With this, a liquid tends to flow to the second base portion 16 lower than the first base portion 15.

For example, when a liquid containing a filtration target is dropped on the first main surface PS1 of the filtration filter 10, the liquid tends to flow from the first base portion 15 toward the second base portion 16. With this, the liquid tends to spread over the entire filtration filter 10, and the filtration area actually used for filtration in the area of the entire filter may be increased.

More specifically, when the liquid is dropped, the liquid droplet existing across the boundary between the first base portion 15 and the second base portion 16 moves toward the second base portion 16 under the influence of gravity because of the height difference, and a flow of the liquid in the direction occurs. Since the flows of the liquid occur from the plurality of first base portions 15 on both sides of the second base portion 16, sandwiching the second base portion 16 to the second base portion 16, the flows along the second base portion 16 intensify with each other. As a result, acceleration occurs in the direction in which the second base portion 16 extends, for example, in the width direction (X direction and/or Y direction), and a liquid spreads. Thus, the filtration area may be increased.

Since the filtration area is increased, the liquid may easily pass through the filtration filter 10. This makes it possible to shorten the filtration time.

There follows equation (1) representing the flow rate per unit filtration area for the filtration filter 10 having the through-holes 13 of which sectional shape is a square.

$$J = N \times π \times {a}^{4} \times ( \frac{Δ P}{128 μ t} ) \qquad ( 1 )$$

Here, J is the flow rate per unit filtration area (m³/m²·s), N is the number of circular tubes per unit filtration area (1/m²), a is length of one side (m) of a square through-hole, ΔP is the differential pressure (Pa) between both ends of the circular tube, μ is the viscosity (Pa·s) of the fluid, and t is the thickness (m) of the through-hole.

Equation (1) indicates that a liquid having spread because of the height difference in the filter base portion 14 tends to pass through the through-holes 13 around the second base portion 16 of a small thickness. As a result, the filtration area is increased, and further, the liquid tends to pass through around the second base portion 16 of a small thickness. This makes it possible to shorten the filtration time. With this, the filtration efficiency may be increased.

The width h2 of the plurality of second base portions 16 is larger than the width h1 of the plurality of first base portions 15. With the configuration above, the liquid further tends to spread over the entire filtration filter 10. As a result, the filtration area may be further increased, and the filtration efficiency may be further increased.

Further, the liquid flowing from the first base portion 15 toward the second base portion 16 tends to accumulate on the second base portion 16. When the filtration target is a cell, the cell is captured by the filtration filter 10 in a state of being immersed in the liquid. As a result, it is possible to suppress a decrease in the activity of the cell captured by the filtration filter 10.

The plurality of first base portions 15 and the plurality of second base portions 16 are provided in a grid pattern. With the configuration above, the filtration efficiency may be further increased.

The plurality of second base portions 16 are periodically arranged. With the configuration above, the liquid further tends to spread over the entire filtration filter 10, and the filtration efficiency may be further increased.

The filtration filter 10 includes the reinforcement layer 17 provided on the first base portion 15. With the configuration above, the mechanical strength of the filtration filter 10 may be increased.

The filtration filter 10 contains at least one of metal and metal oxide as a main component. With the configuration above, the filtration efficiency may be further increased.

Note that, in Embodiment 1, an example in which the filtration filter 10 includes the filter portion 11 and the frame portion 12 has been described, but the present invention is not limited thereto. For example, it is not necessary that the filtration filter 10 includes the frame portion 12. The frame portion 12 is not an essential constituent.

In Embodiment 1, an example in which the second main surface PS2 of the filtration filter 10 is flat has been described, but the present invention is not limited thereto. For example, it is not necessary that the second main surface PS2 of the filtration filter 10 is flat. A step may be provided on the second main surface PS2 of the filtration filter 10.

In Embodiment 1, an example in which the plurality of first base portions 15 and the plurality of second base portions 16 are each provided in a grid pattern has been described, but the present invention is not limited thereto. It is sufficient that the plurality of first base portions 15 and the plurality of second base portions 16 are provided to be able to define the plurality of through-holes 13. For example, the plurality of first base portions 15 and the plurality of second base portions 16 may be provided to obliquely intersect with each other.

In Embodiment 1, an example in which the plurality of second base portions 16 are periodically arranged has been described, but the present invention is not limited thereto. It is sufficient that the plurality of second base portions 16 are provided between the plurality of first base portions 15. The plurality of second base portions 16 may be provided at random.

In Embodiment 1, an example in which the width h2 of the second base portion 16 is larger than the width h1 of the first base portion 15 has been described, but the present invention is not limited thereto. For example, the width h2 of the second base portion 16 may be equal to or less than the width h1 of the first base portion 15.

In Embodiment 1, an example in which the reinforcement layer 17 is provided on the first base portion 15 has been described, but the present invention is not limited thereto. It is not necessary that the filtration filter 10 includes the reinforcement layer 17. The reinforcement layer 17 is not an essential constituent.

In Embodiment 1, an example in which a liquid is a cell suspension and a filtration target is a cell has been described, but the present invention is not limited thereto.

### Examples

Filtration was performed using Example 1 and Comparative Example 1.

In Example 1, the filtration filter 10 of Embodiment 1 was used. In Comparative Example 1, a filtration filter including no second base portion 16, that is, a filtration filter including only the first base portion 15 was used. The conditions of the filtration filters of Example 1 and Comparative Example 1 is listed in Table 1.

TABLE 1

| | | Comparative |
|---|---|---|
| | Example 1 | Example 1 |
| Outer diameter of filtration | 33 | mm |
| filter 10 | | |
| Diameter of filter portion 11 | 28 | mm |
| Shape of filter base portion 14 | Grid | |
| Shape of through-hole 13 | Square | |
| Dimension of through-hole 13 | 10 μm in one side | |
| Width h1 of the first filter base | 4.1 | μm |
| portion 15 | | |
| Thickness t1 of the first filter | 1.6 | μm |
| base portion 15 | | |
| Interval P1 between the first | 14.1 | μm |
| filter base portions 15 | | |
| Width h2 of the second filter | 6.8 μm | — |
| base portion 16 | | |
| Thickness t2 of the second filter | 1.0 μm | — |
| base portion 16 | | |
| Interval P2 between the second | 9 mm | — |
| filter base portions 16 | | |

In the filtration filter 10 of Example 1, a height difference (unevenness) is formed by the plurality of first base portions 15 and the plurality of second base portions 16 on the first main surface PS1 on which a filtration target is captured. In the filtration filter of Comparative Example 1, the first main surface PS1 is formed in a flat shape.

Each of the filtration filters of Example 1 and Comparative Example 1 was sandwiched and held by a resin-made holder, and filtration was performed by dripping a liquid containing a filtration target. Specifically, in order to evaluate the filtration characteristics of the filtration filters of Example 1 and Comparative Example 1, a liquid passage test was performed.

First, each of a resin-made holder holding the filtration filter 10 of Example 1 and a resin-made holder holding the filtration filter of Comparative Example 1 was installed at an opening of a 50-ml centrifuge tube. Next, 2 ml of 70% (v/v) ethanol was dripped to each of the filtration filter 10 of Example 1 and the filtration filter of Comparative Example 1, and then 10 ml of Milli-Q water was dripped.

Thereafter, the resin-made holder holding the filtration filter 10 of Example 1 and the resin-made holder holding the filtration filter of Comparative Example 1 were installed in respective 50-ml centrifuge tubes, and 5 ml of a test liquid was introduced into each of the filtration filter 10 of Example 1 and the filtration filter of Comparative Example 1.

The time until all of the 5-ml test liquid passed through the filtration filter (hereinafter, referred to as filtration time), the quantity of the liquid collected in the 50-ml centrifuge tube (hereinafter, referred to as filtration quantity), and the concentration of the number of cells in the collected liquid was measured. The upper limit of the filtration time was set to five minutes. When all of the 5-ml test liquid did not pass through the filtration filter after five minutes, it was determined that the filtration filter was clogged. Four types of test liquids were used: pure water, phosphate buffered saline (hereinafter, referred to as PBS), culture medium, and cell suspension of HL-60 cells (1.86×10⁶ cells/ml). Further, for each test liquid, an image of the filtration filter after the liquid passage test was taken at a magnification of 1 or 2 under a stereo-microscope (Shimadzu Corporation, model number: STZ-16) after filtration. Then, using image analysis software Image J (US National Institutes of Health), the area where the test liquid spread (hereinafter, filtration area) was roughly estimated. The evaluation results are listed in Table 2.

TABLE 2

| | | Test liquid | | | |
|---|---|---|---|---|---|
| | | | | | Cell |
| | | | | | suspension |
| Filtration | Evaluation | Pure | | Culture | of HL-60 |
| filter used | item | water | PBS | medium | cells |
| Example 1 | Filtration time | 9 | 11 | 10 | 16 |
| | (s) | | | | |
| | Filtration | 5 | 5 | 5 | 4.7 |
| | quantity (ml) | | | | |
| | Concentration | — | — | — | 9.58 × 10⁵ |
| | of the number | | | | |
| | of cells | | | | |
| | (cells/ml) | | | | |
| | Filtration area | 188.7 | 203.9 | 178.5 | 183.3 |
| | (mm²) | | | | |
| Comparative | Filtration time | 15 | 17 | 18 | 20 |
| Example 1 | (s) | | | | |
| | Filtration | 5 | 5 | 4.9 | 4.3 |
| | quantity (ml) | | | | |
| | Concentration | | | | |
| | of the number | | | | |
| | of cells | — | — | — | 2.74 × 10⁵ |
| | (cells/ml) | | | | |
| | Filtration area | | | | |
| | (mm²) | 136.9 | 112.4 | 121.8 | 103.7 |

As listed in Table 2, it is found that in Example 1, the filtration time is shorter, the concentration of the number of cells in the collected liquid is higher, and the filtration area is larger than in Comparative Example 1.

While the present invention has been sufficiently described in connection with the preferred embodiments thereof with reference to the accompanying drawings, various changes and modifications are apparent to those skilled in the art. Such variations and modifications are to be understood as being included within the scope of the present invention as defined by the appended claims unless departing therefrom.

The filtration filter of the present invention is useful for filtering a cell suspension, for example.

### REFERENCE SIGNS LIST

10 FILTRATION FILTER
11 FILTER PORTION
12 FRAME PORTION
13 THROUGH-HOLE
14 FILTER BASE PORTION
15 FIRST BASE PORTION
16 SECOND BASE PORTION
17 REINFORCEMENT LAYER
21 SUBSTRATE
22 COPPER THIN FILM
23 INTERMEDIATE LAYER
24 RESIST FILM
25, 26, 27 PdNi PLATING FILM

1. A filtration filter for filtering a liquid containing a filtration target, the filtration filter comprising:
a filter base portion defining a plurality of through-holes,
wherein the filter base portion includes a plurality of first base portions and a plurality of second base portions that are thinner than the plurality of first base portions, and
each of the plurality of second base portions are between the plurality of first base portions.

2. The filtration filter according to claim 1, wherein a width of the plurality of second base portions is larger than a width of the plurality of first base portions.

3. The filtration filter according to claim 2, wherein the width of the plurality of second base portions is 1.1 times to 4 times the width of the plurality of first base portions.

4. The filtration filter according to claim 1, wherein the plurality of first base portions and the plurality of second base portions are in a grid pattern.

5. The filtration filter according to claim 1, wherein the plurality of second base portions are periodically arranged.

6. The filtration filter according to claim 1, further comprising:
a reinforcement layer on the first base portion.

7. The filtration filter according to claim 1, wherein the filter base portion comprises at least one of a metal and a metal oxide as a main component thereof.

8. The filtration filter according to claim 1, wherein a first interval between the plurality of second base portions is larger than a second interval between the plurality of first base portions.

9. The filtration filter according to claim 1, wherein the filter base portion includes a first main surface and a second main surface, the plurality of through-holes extending between the first main surface and the second main surface, and the second main surface being substantially flat.

10. The filtration filter according to claim 1, wherein a thickness of the second base portion is 0.25 times to 0.95 times a thickness of the first base portion.

11. The filtration filter according to claim 1, wherein a ratio of an area occupied by the plurality of second base portions is smaller than a ratio of an area occupied by the plurality of first base portions.

12. The filtration filter according to claim 11, wherein the area occupied by the plurality of second base portions is 0.001 times to 0.8 times the area occupied by the plurality of first base portions.
